(19) United States
(12) Patent Application Publication
(10) Pub. No.: US 2002/0127588 A1
(43) Pub. Date: Sep. 12, 2002

(54) Hybridization device

(76) Inventors: Keiichi Sato, Kanagawa (JP); Toshiki Morita, Kanagawa (JP)

Correspondence Address: Pillsbury Winthrop LLP, Suite 200, 11975 El Camino Real, San Diego, CA, 92130, US

(21) Appl. No.: 10/077,096

(22) Filed: Feb. 14, 2002

(30) Foreign Application Priority Data
Mar. 6, 2001 (JP) 62418/2001

(51) Int. Cl. C12Q 1/68; C12M 1/34

(52) U.S. Cl. 435/6; 435/287.2

(57) ABSTRACT

The present invention has an objective of providing a hybridization device which anyone can easily and steadily set up, and with which evaporation of a sample solution can be minimized.

A silicone-rubber-based sheet 2 has a hydrophilic region 3 and a hydrophobic region 4 surrounding the hydrophilic region 3, the hydrophilic region 3 corresponding to a region of a substrate (not shown) where DNA is fixed. Accordingly, hybridization reaction that takes long time can be carried out stably with a small amount of sample solution even without a sealing case or water for preventing the sample solution from evaporating.

### PRIORITY INFORMATION

This application claims priority to Japanese Application Serial No. 2001-62418, filed Mar. 6, 2001.

### BACKGROUND OF THE INVENTION

The present invention relates to a hybridization device for determining the presence or absence of a sequence of interest in a sample biopolymer through hybridization reaction between the sample biopolymer and a probe biopolymer.

Conventionally, hybridization methods that use nucleic acids or proteins with known sequences as probes are often employed in order to identify and/or fractionate a molecular in an organism, particularly in order to detect DNA of interest or the presence of genetic DNA. Specifically, a DNA chip is used which is a slide glass on which probe DNAs are fixed. First, a solution containing fluorescence-labeled sample DNA is dropped on the slide glass having probe DNAs fixed thereon. Then, the slide glass is covered with a cover glass to allow hybridization reaction. The sample DNA that binds to the probe DNA stays with the probe DNA. After washing the slide glass, the fluorescent substance labeling the remaining fixed sample DNA is excited with excitation light from a light source to detect the emitted fluorescence, thereby detecting the hybridized sample DNA.

As a preferable hybridization device for the above-described hybridization reaction, a cassette used for a hybridization reaction thermostat (CHBIO) is known (Hitachi Software Engineering Co. Ltd.).

FIG. 6 is a perspective view showing a structure of a conventional hybridization device. This hybridization device mainly consists of a case 21 and a tray/cap unit 22 (a housing and a housed member). The tray/cap unit 22 is provided with a tray 24 for holding a slide glass and a packing 25 made of silicone rubber or the like for enhancing sealing. The case 21 and the tray/cap unit 22 can be united with a locking unit 23, whereby the case 21 and the tray/cap unit 22 enclose a sealed space together.

FIG. 7 is a perspective view showing the appearance of the united hybridization device. In the figure, the same reference numerals denote the same components as FIG. 6. The actual dimensions of the united device are 94×41×13 mm³.

The cover glass is used to carry out efficient hybridization reaction with a less amount of sample solution. However, since the cover glass is extremely thin and light, placing and fixing the cover glass on the sample solution at a predetermined position of the slide glass requires certain experience and technique. In the worst case, the glass cover may be broken and the precious sample may be wasted.

Since the amount of the sample solution is small as described above while the hybridization reaction takes place at a relatively high temperature, the sample solution may evaporate. As a result, the component contained in the solution remaining after the evaporation of the sample solution is hard to wash off and likely to stay on the glass slide, resulting as noise upon analysis.

In view of the above-described problems, the present invention has an objective of providing a hybridization device which anyone can easily and steadily set up and where the amount of an evaporated sample solution is minimized.

### SUMMARY OF THE INVENTION

Thus, the hybridization device of the present invention comprises a sheet having a hydrophilic surface region and a hydrophobic surface region surrounding the hydrophilic region, the hydrophilic surface region facing a probe-biopolymer-fixed region of a substrate when the sheet and the probe-biopolymer-fixed substrate are arranged in layers.

The hybridization device of the invention comprises a sheet having a hollowed region and a region surrounding the hollowed region, the hollowed region facing a probe-biopolymer-fixed region of a substrate when the sheet and the probe-biopolymer-fixed substrate are arranged in layers.

By making the surface of the hollowed region of the sheet hydrophilic while the surface of the region surrounding the hollowed region hydrophobic, the hollowed region can be filled with a sample solution.

By making the sheet from a material that has affinity with the substrate, the sheet and the substrate can adhere to each other to enhance sealing for enclosing the sample solution.

The substrate on which probe biopolymers are fixed may be made of glass, plastic, metal or the like. The material of the sheet needs to be properly selected according to the material of the substrate. For example, when the substrate is made of glass, the sheet is preferably made of silicone rubber which has adhesiveness with glass, elasticity, hydrophobic property and no biochemical activity.

The above-described sheet may be slightly larger than the substrate so that the corner of the sheet can easily be pinched.

The hybridization device of the invention comprises a substrate fixed with a probe biopolymer and the above-described sheet.

### BRIEF DESCRIPTION OF THE DRAWINGS

FIG. 1 is a schematic view showing a structure of a hybridization device according to a first embodiment of the present invention;

FIG. 2 is a schematic view showing an appearance of a hybridization apparatus incorporating the hybridization device of the first embodiment of the invention;

FIG. 3 is a schematic view for illustrating a method for producing the hybridization device according to the first embodiment of the invention;

FIG. 4 is a schematic view showing a structure of a hybridization device according to a second embodiment of the present invention;

FIG. 5 is a schematic view showing a structure of a hybridization device according to a third embodiment of the present invention;

FIG. 6 is a schematic view showing a structure of a conventional hybridization device; and

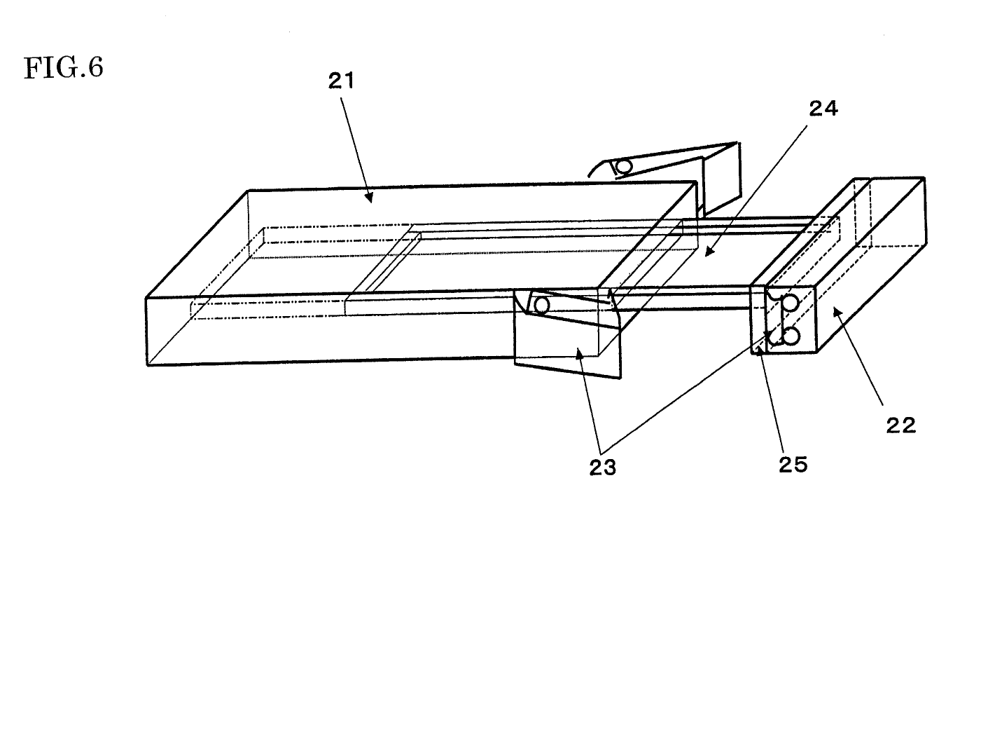

FIG. 7 is a schematic view showing the appearance of the united hybridization device.

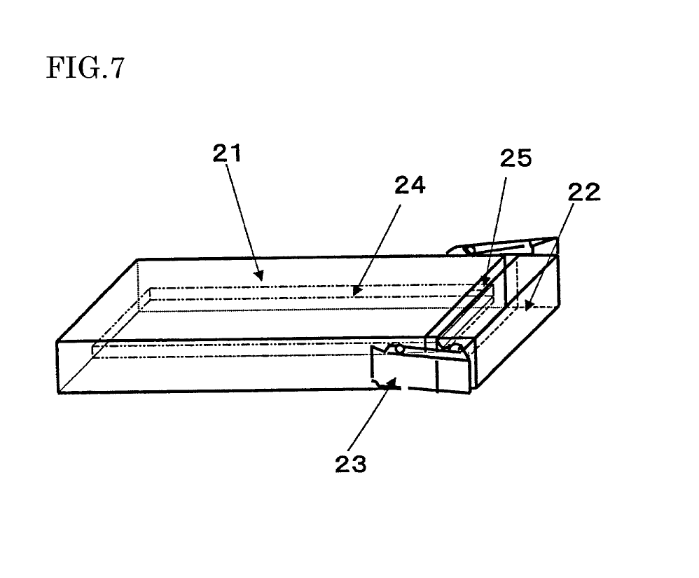

### PREFERRED EMBODIMENTS OF THE INVENTION

Hereinafter, preferred embodiments of the present invention will be described in details with reference to the accompanying drawings.

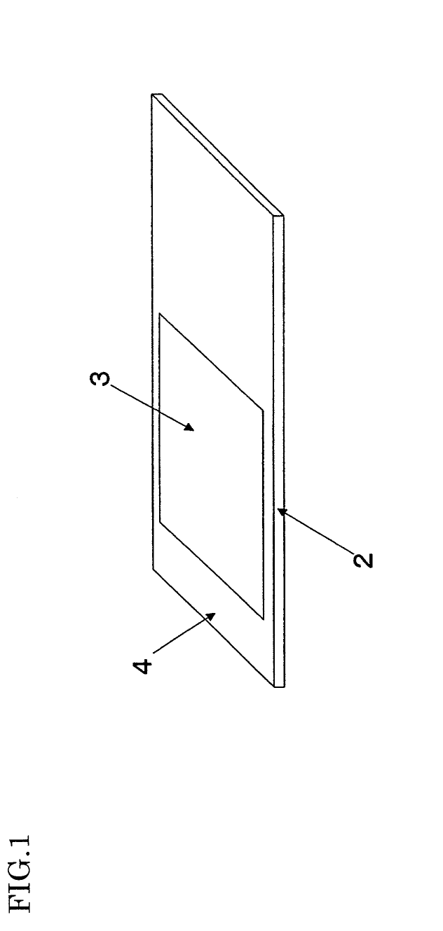

FIG. 1 is a schematic view showing a structure of a hybridization device according Embodiment 1 of the present invention. A silicone-rubber-based sheet 2 has a hydrophilic region 3 and a hydrophobic region 4. The hydrophilic region 3 is a region that faces a DNA-fixed (generally biopolymer-fixed) region of a substrate (not shown) and the hydrophobic region 4 is a surface region of the sheet 2 surrounding the hydrophilic region 3. The thickness of the sheet 2 is about 1 mm. The substrate on which the probe biopolymers are fixed is made of glass, plastic, metal or the like. The dimensions of the slide glass commonly used as the substrate is 76×26 mm² according to the Japanese standard, 3×1 inch² (25.4 mm²) according to the American standard, and 25×75 mm² according to the European standard. Silicone rubber is generally hydrophobic and glass is hydrophilic.

By way of example, a method for performing hybridization reaction using the silicone-rubber-based sheet 2 and the slide glass substrate will be described.

(1) A sample solution is dropped on the hydrophilic region 3 of the sheet 2 such that the sample solution slightly stands above the surface of the hydrophilic region. Since the hydrophilic region 3 is surrounded by the hydrophobic region 4, the sample solution selectively stays on the hydrophilic region 3.

(2) The slide glass is carefully placed down with the fixed DNAs facing down. Since silicone rubber has good affinity with glass, the silicone-rubber-based sheet 2 has a sufficient level of adhesiveness with the slide glass to seal the sample solution.

(3) The device is maintained at a relatively high constant temperature to allow hybridization reaction. During the course of reaction, a slight amount of sample solution on the hydrophobic region 4 will evaporate, which will enhance adhesiveness and thus sealing.

(4) The sheet 2 is peeled off from the slide glass. By using an elastic material such as silicone rubber as the sheet 2, it is easier to separate the hydrophobic region 4 of the sheet 2 from the slide glass. Since the sample solution remains on the hydrophilic region 3, the hydrophilic region 3 of the sheet 2 is much easier to be separated from the slide glass. The peeling can further be facilitated by making the size of the sheet 2 larger than the size of the slide glass so that the corner of the sheet 2 can be pinched.

(5) Thereafter, similar to the conventional technique, the slide glass is washed, excited with excitation light and subjected to detection of the emitted fluorescence.

According to the above-described hybridization reaction, hybridization reaction that takes long time can be carried out with a small amount of sample solution in a stable manner without using a sealing case or water for preventing the sample solution from evaporating.

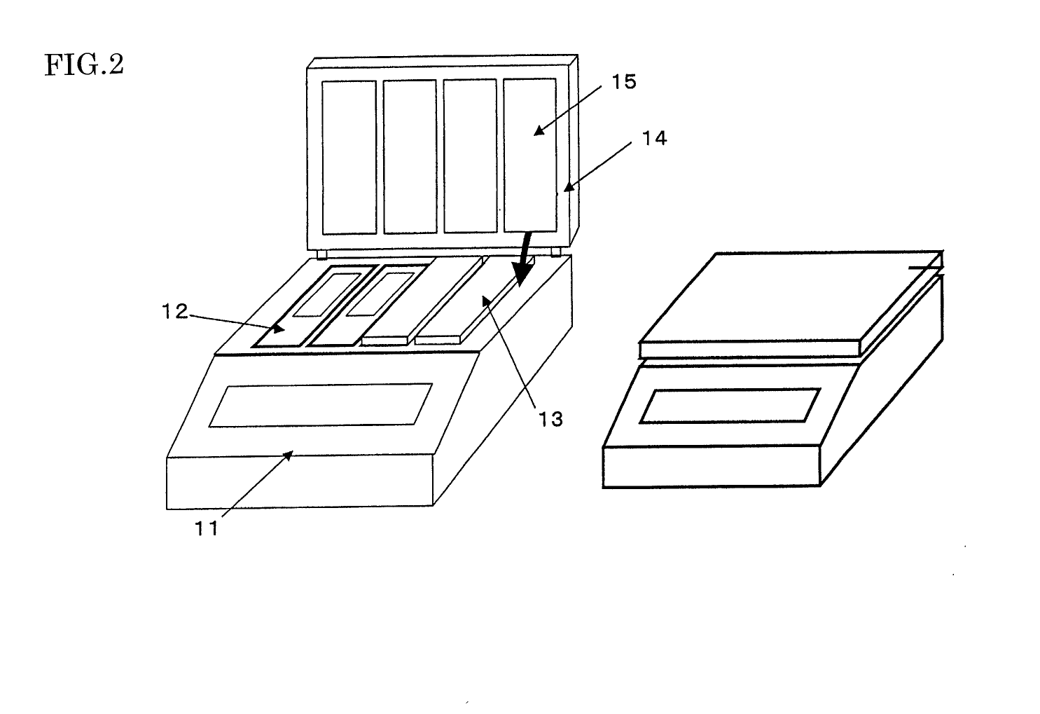

FIG. 2 is a schematic view showing an appearance of a hybridization reaction apparatus incorporating the hybridization device according to Embodiment 1 of the present invention. The hybridization apparatus itself is a commonly used apparatus. The hybridization reaction using this apparatus can take place as follows. First, sheets 12 are placed on the apparatus 11, and then substrates 13 are placed on the sheets 12. Although the structure of the devices already gives good adhesiveness as described above, an apparatus lid 15 with a packing 14 may further be used to maintain a constant temperature to allow hybridization reaction. In this manner, the device of the invention can be applied to a commonly used hybridization apparatus.

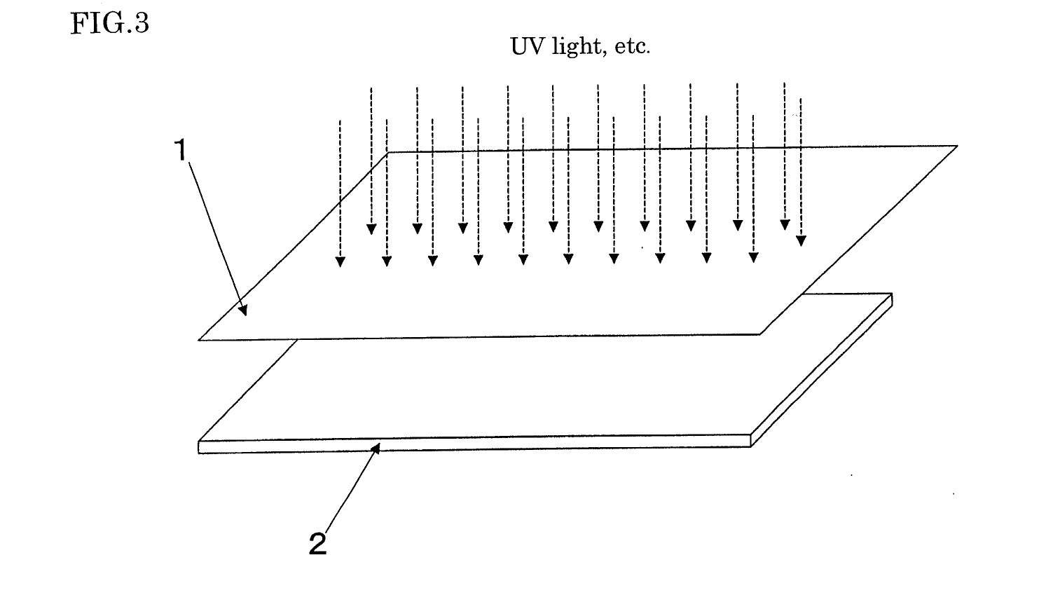

FIG. 3 is a schematic view for illustrating a method for producing the hybridization device according to Embodiment 1 of the invention. Herein, a predetermined region of a commonly used silicone rubber sheet 2 is selectively given affinity via a photocatalyst technique.

(1) A thin film containing a photocatalyst semiconductor material is formed over the entire surface of the silicone rubber sheet 2. The photocatalyst semiconductor material is selected from a group consisting of TiO₂, ZnO, SnO₂, SrTiO₃, WO₃, Bi₂O₃ and Fe₂O₃ (for further detail, see Japanese Patent No. 2756474).

(2) The sheet 2 is irradiated with UV light via a mask 1 that allows the UV light to pass through at a selected region corresponding to the hydrophilic region 3. Accordingly, the hydrophilic region 3 of the formed photocatalyst semiconductor thin film is irradiated with UV light, thereby converting the photocatalyst semiconductor thin film formed on the hydrophilic region 3 into a hydrophilic region. Silicone rubber is hydrophobic and remains hydrophobic even if the above-described photocatalyst semiconductor material is formed thereon as long as the photocatalyst semiconductor material is not irradiated with UV light. Thus, the hydrophobic region 4 remains hydrophobic.

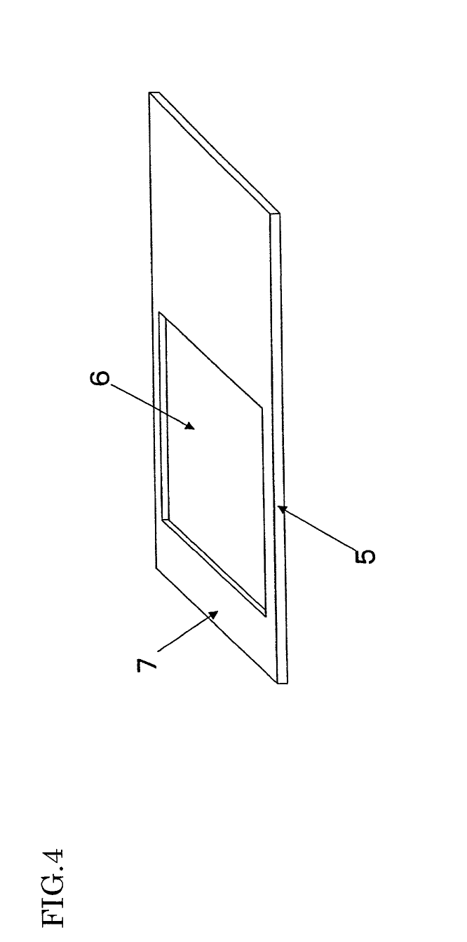

FIG. 4 is a schematic view showing a structure of a hybridization device according to Embodiment 2 of the present invention. According to the present embodiment, a silicone-rubber-based sheet 5 is produced by forming a hollow 6 in the surface of the sheet corresponding to a DNA-fixed (generally biopolymer-fixed) region of a slide glass (not shown), and then making the surface of the hollow 6 hydrophilic. The region around the hollow 6 remains hydrophobic as a hydrophobic region 7. As an alternative of Embodiment 2, a region surrounding the hollow 6 may be hydrophilic as long as it is further surrounded by a hydrophobic region 7 for enclosing the sample solution.

Since the hollow 6 is formed according to Embodiment 2, a larger amount of sample solution can be enclosed, which is preferable for a long-time hybridization reaction.

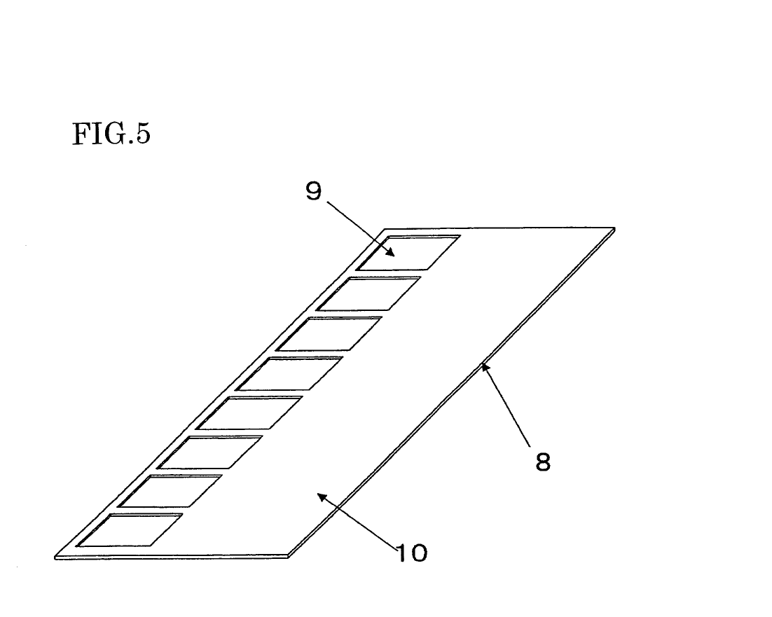

FIG. 5 is a schematic view showing a structure of a hybridization device according to Embodiment 3. According to this embodiment, a silicone-rubber-based sheet 8 has a plurality of hollows 9 on its surface corresponding to DNA-fixed (generally biopolymer-fixed) regions of a plurality of slide glasses. The surfaces of the hollows 9 are made hydrophilic. The region surrounding the hollows 9 remains hydrophobic as a hydrophobic region 10.

According to Embodiment 3, hybridization reaction can be carried out for a plurality of slide glasses by using the sheet 8, thereby simplifying manipulation.

The present invention is not limited to the above-described embodiments.

The present invention also contemplates a hybridization device comprising a combination of a slide glass fixed with probe biopolymers and the above-described sheet.

Instead of using a photocatalist semiconductor material, the hydrophilic property can be achieved by applying a hydrophilic coating.

The material of the sheet is not limited to silicone rubber, and can be any material that can have both hydrophilic and hydrophobic regions on its surface and optionally a hollow in its surface. It is more preferable that the material has adhesiveness with the substrate, elasticity, hydrophobic property and no biochemical activity.

Although the region other than the hydrophilic regions of the sheet is entirely hydrophobic, this is not necessary as long as the hydrophilic region is surrounded by a hydrophobic region.

According to the present invention, the hybridization device can be set up easily and steadily. In addition, evaporation of the sample solution can be minimized.

What is claimed is:

1. A hybridization device comprising a sheet having a hydrophilic surface region and a hydrophobic surface region surrounding the hydrophilic region, the hydrophilic surface region facing a probe-biopolymer-fixed region of a substrate when the sheet and the probe-biopolymer-fixed substrate are arranged in layers.

2. A hybridization device comprising a sheet having a hollowed region and a region surrounding the hollowed region, the hollowed region facing a probe-biopolymer-fixed region of a substrate when the sheet and the probe-biopolymer-fixed substrate are arranged in layers.

3. A hybridization device according to claim 2, wherein the surface of the hollowed region of the sheet is hydrophilic while the surface of the region surrounding the hollowed region is hydrophobic.

4. A hybridization device according to claim 1, wherein the sheet is made of a material that has affinity with the substrate.

5. A hybridization device according to claim 4, wherein the sheet is made of silicone rubber.

6. A hybridization device according to claim 1, wherein the sheet is slightly larger than the substrate.

7. A hybridization device, comprising a substrate fixed with a probe biopolymer and the sheet of claim 1.
